(19) United States
(12) Patent Application Publication
(10) Pub. No.: US 2007/0218018 A1
(43) Pub. Date: Sep. 20, 2007

(54) SENSITIVITY RELIEF GEL

(75) Inventor: Jeff MacDonald, Pomona, CA (US)

Correspondence Address: DISCUS DENTAL IMPRESSIONS, INC., 8550 HIGUERA STREET, CULVER CITY, CA, 90232, US

(73) Assignee: DISCUS DENTAL IMPRESSIONS, INC., Culver City, CA (US)

(21) Appl. No.: 11/686,298

(22) Filed: Mar. 14, 2007

Related U.S. Application Data
(60) Provisional application No. 60/783,190, filed on Mar. 15, 2006.
(60) Provisional application No. 60/892,859, filed on Mar. 4, 2007.

(51) Int. Cl. A61K 8/21

(52) U.S. Cl. 424/52

(57) ABSTRACT

The present invention provides a sensitivity relief gel preferably for use following a dental treatment such as a whitening session. The relief gel contains at least one desensitizing agent, at least one source of fluoride and at least one gelling agent. The relief gel has reduced gumminess and is easy to apply and remove. The gelling agent is, for example, a non-ionic surfactant, more for example, a block copolymer with hydrophobic and hydrophilic blocks. The present invention further provides for a method of using the sensitivity relief gel, for example, after a bleaching process.

### CROSS REFERENCE TO RELATED CASES

This application claims the benefit of an U.S. provisional patent application Nos. 60/783,190, filed Mar. 15, 2006; and 60/892,859, filed Mar. 4, 2007; the contents of which are hereby incorporated by reference in their entirety.

### FIELD OF THE INVENTION

This invention relates to tooth sensitivity relief gels. More particularly, this invention relates to sensitivity relief gels suitable for office or home use.

### BACKGROUND OF THE INVENTION

Dental procedures may be necessitated by tooth stains and/or tooth decays. Sensitive teeth may develop after undergoing a dental procedure. Sometimes, sensitive teeth may develop even without undergoing any dental procedure.

A tooth is composed of an inner dentin layer and an outer hard enamel that is coated with a protective layer called the acquired pellicle. The enamel layer is composed of hydroxyapatite crystals that create a somewhat porous surface. The pellicle or the enamel can become stained or discolored. It is believed that the porous nature of the enamel layer is what allows staining agents and discoloring substances to permeate the enamel and discolor the tooth.

Many substances that a person's teeth confront or come in contact with on a daily basis can “stain” or reduce the “whiteness” of one's teeth. In particular, food products, tobacco products and fluids such as tea and coffee that one consumes tend to stain one's teeth. These staining and discoloring substances can then permeate the enamel and causing noticeable discoloration of one's teeth. One solution to this problem is through tooth bleaching.

Whitening may be accomplished in an in-office treatment or home treatment. The amount of whitening obtained during tooth bleaching is dependent upon (1) the length of time each day the tray is worn; (2) the number of days the tray is worn; (3) the susceptibility of the teeth to the bleaching agent and (4) the concentration of active peroxides. For maximum whitening, an accelerated treatment time of approximately 18-20 hours per day is recommended.

Some dentifrices, like toothpastes, gels, and powders, and whitening strips, contain active oxygen or hydrogen peroxide liberating bleaching agents including peroxides, percarbonates and perborates of the alkali and alkaline earth metals or complex compounds containing hydrogen peroxide. These may also be used in whitening tooth.

One concern with some bleaching compositions is that prolonged treatment with highly concentrated bleaching agents present in the composition may contribute to tooth sensitivity following treatment. Even treatments with compositions not known to increase tooth sensitivity in most patients might still cause sensitivity in patients more prone to such sensitivity tendencies.

Although dentifrices such as tooth pastes are available for sensitivity relief, they might not be desirable for use after a whitening session as they normally contain abrasives that might also cause irritation. Even those that are recommended for use specifically after such bleaching sessions have deficiencies such as gumminess, and difficulty of application and removal.

Therefore, there exists a need for a dental gel with sensitivity relief designed for sensitivities, either caused, for example, by bleaching processes or other dental procedures, or even without any treatments, without the above mentioned deficiencies.

### SUMMARY OF INVENTION

The present invention discloses a sensitivity relief gel for use following a dental treatment or on sensitive teeth.

In one embodiment, the sensitivity relief gel includes a viscous gel including:

at least one de-sensitizing substance;

at least one source of fluoride ion at a concentration of up to about 1500 ppm; and

at least one non-ionic surfactant comprising a block copolymer having hydrophobic and hydrophilic blocks; wherein said gel has a storage modulus (E′) of about at least 250,000 Pa (Pascals) at low frequencies of about 0.01 Hz, at a strain of about 0% to about 10%.

The temperature of measurement may be between about 20° C. to about 40° C., more for example, from about 21° C. to about 37° C.

In another embodiment, the sensitivity relief gel includes a viscous gel including:

at least one de-sensitizing substance;

at least one source of fluoride ion at a concentration of up to about 1500 ppm; and

at least one non-ionic surfactant comprising a block copolymer having hydrophobic and hydrophilic blocks;

wherein said gel has a storage modulus that stays substantially constant from a frequency of about 0.01 Hz to a frequency of about 50 Hz, at a strain of about 0% to about 10%.

In a further embodiment, the sensitivity relief gel includes a viscous gel including at least one de-sensitizing substance;

at least one source of fluoride ion at a concentration of up to about 1500 ppm; and

at least one non-ionic surfactant comprising a block copolymer having hydrophobic and hydrophilic blocks; wherein said gel has a storage viscosity of more than about 20,000 Pa·s (Pascal·seconds) at frequencies of about 0.1-0.5 Hz, at a strain of about 0% to about 10%.

In one aspect of the invention, the sensitivity relief gel is substantially free of abrasives.

In another aspect of the invention, the relief gel is easy to apply and remove.

In a further aspect of the invention, the sensitivity relief gel has reduced gumminess.

The present invention also provides for a package including a whitening composition and a viscous gel.

In one embodiment, the package includes:

a whitening composition including at least one source of peroxide; and

a viscous gel including:
at least one de-sensitizing substance;
at least one source of fluoride ion at a concentration of up to about 1500 ppm; and
at least one non-ionic surfactant comprising a block copolymer having hydrophobic and hydrophilic blocks;
   wherein said gel has a storage modulus (E′) of about at least 250,000 Pa (Pascals) at low frequencies of about 0.01 Hz, at a strain of about 0% to about 10%.

In another embodiment, the package includes:

a whitening composition including at least one source of peroxide; and

a viscous gel including:
at least one de-sensitizing substance;
at least one source of fluoride ion at a concentration of up to about 1500 ppm; and
at least one non-ionic surfactant comprising a block copolymer having hydrophobic and hydrophilic blocks;
   wherein said gel has a storage modulus that stays substantially constant from a frequency of about 0.01 Hz to a frequency of about 50 Hz, at a strain of about 0% to about 10%.

In a further embodiment, the package includes:

a whitening composition including at least one source of peroxide; and

a viscous gel including at least one de-sensitizing substance;

at least one source of fluoride ion at a concentration of up to about 1500 ppm; and

at least one non-ionic surfactant comprising a block copolymer having hydrophobic and hydrophilic blocks;

wherein said gel has a storage viscosity of more than about 20,000 Pa·s (Pascal·seconds) at frequencies of about 0.1-0.5 Hz, at a strain of about 0% to about 10%.

The viscous gel may be used following the application of the whitening composition.

In one aspect, the whitening composition may be packaged in a separate container or package inside the main package. The container or package may include a syringe, a double-barrel syringe, a squeezable tube, a pumpable container, a multiple-compartment container, a dental pen, a tray or similar.

In another aspect, the package may contain more than one whitening package.

In a further aspect, the viscous gel may be packaged in a separate container including a syringe, a double-barrel syringe, a squeezable tube, a pumpable container, a multiple-compartment container, a dental pen, a tray or similar.

In one embodiment, the package may be a bubble-like container. In another embodiment, the package may be a box-like container. In one aspect, the container may be fitted with a band having personalization capability. In another aspect, the container may be fitted with a band capable of different color schemes.

The present invention additionally provides for a method of using the sensitivity relief gel, comprising the steps of:
(1) providing a bleaching gel comprising at least one peroxide to effect teeth bleaching; and
(2) applying a sensitivity relief gel comprising:

a viscous gel comprising from about 2.5% to about 6% by weight of at least one de-sensitizing alkali metal salt or an amorphous alkaline phosphate compound;

at least one source of fluoride ion at a concentration of up to about 1500 ppm; and

at least one non-ionic surfactant comprising a block copolymer of hydrophobic and hydrophilic blocks;

wherein said gel has a storage modulus (E′) of from about at least 250,000 Pa (Pascals) at low frequencies of less than about 0.01 Hz, at a strain of about 0% to about 10%.

The temperature of measurement may vary between about 20° C. to about 40° C., more for example, from about 21° C. to about 37° C.

### BRIEF DESCRIPTION OF THE FIGURES

FIG. 1a shows a modulus scan at 21.5° C., a dynamic load of 110 mN, a static load of 100 mN and a strain at 10%;

FIG. 1b shows a viscosity scan at 21.5° C., a dynamic load of 110 mN, a static load of 100 mN and a strain at 10%;

FIG. 2a shows a modulus scan at 37° C., a dynamic load of 110 mN, a static load of 100 mN and a strain at 10%;

FIG. 2b shows a viscosity scan at 37° C., a dynamic load of 110 mN, a static load of 100 mN and a strain at 10%;

FIG. 3a shows a modulus scan at 37° C., a dynamic load of 110 mN, a static load of 100 mN and a strain at 2.5%;

FIG. 4a shows a modulus scan at 37° C., a dynamic load of 110 mN, a static load of 100 mN and a strain at 0%;

FIG. 4b shows a modulus scan at 21.5° C., a dynamic load of 110 mN, a static load of 100 mN and a strain at 0%; and

FIGS. 5a and 5b show an example of an oscillating rheometer;

FIGS. 6, 6a, 6b, 6c, and 6d illustrate embodiments of dental trays and coatings of the present invention;

FIGS. 7a, 7b, and 7c illustrate embodiments of packages of the present invention.

### DETAILED DESCRIPTION OF THE EMBODIMENTS

The detailed description set forth below is intended as a description of the presently exemplified composition provided in accordance with aspects of the present invention and is not intended to represent the only forms in which the present invention may be prepared or utilized. The description sets forth the features and the steps for preparing and using the compositions of the present invention. It is to be understood, however, that the same or equivalent functions and ingredients incorporated in the tooth bleaching compositions may be accomplished by different embodiments that are also intended to be encompassed within the spirit and scope of the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the exemplified methods, devices and materials are now described.

All publications mentioned herein are incorporated herein by reference for the purpose of describing and disclosing, for example, the compositions and methodologies that are described in the publications which might be used in connection with the presently described invention. The publications listed or discussed above, below and throughout the text are provided solely for their disclosure prior to the filing date of the present application. Nothing herein is to be construed as an admission that the inventors are not entitled to antedate such disclosure by virtue of prior invention.

As noted above, teeth bleaching is a very popular way to remove stains and whiten teeth. The amount of whitening obtained during tooth bleaching process is generally dependent upon (1) the length of time the tooth/teeth is/are in contact with the whitening agent; (2) the number of days the treatment is carried out; (3) the susceptibility of the teeth to the bleaching agent and (4) the concentration of active peroxides. For maximum whitening, a long treatment time with a highly concentrated bleaching composition is generally recommended. However, such prolonged treatments tend to lead to the development of tooth sensitivity following treatment. Even bleaching gels or processes that do not require such long process times, such as the gel compositions disclosed in co-pending patent application Ser. No. 11/271,283 titled “Dental Whitening Systems”, filed Nov. 9, 2005, and assigned to the common assignee, may still lead to the development of tooth sensitivity in some patients that are prone to such sensitivity.

The present inventors have invented a sensitivity relief gel suitable for use on sensitive teeth or following dental treatments, for example, after a bleaching process, having reduced gumminess, better ability to stay on the teeth or tooth once applied, increase ease of application and ease of removal.

In one exemplary embodiment, the gel includes at least one de-sensitizing salt, at least one source of fluoride and at least one non-ionic surfactant.

The desensitizing salts may include alkali metal salt including a potassium salt, a sodium salt, a lithium salt or mixtures thereof; alkaline metal salts including calcium salts and strontium salts; or mixtures thereof. The salt is, for example, water soluble or at least sparingly water soluble, and the appropriate anions may be chosen to facilitate in their solubility. For example, the appropriate anions may include chlorides, carbonates, nitrates, saccharins, phosphates or mixtures thereof. For example, the alkali metal salt may include at least one potassium salt, or sodium salt, and more for example, potassium nitrate, sodium nitrate, potassium chloride, potassium bicarbonate or sodium saccharin.

The alkali metal salts are, for example, present up to about 6% by weight of the gel, more for example, from about 3% to about 5% by weight of the gel, even more for example, up to about 4 percent by weight, and even more for example, up to about 3 percent by weight.

The alkaline metal salts may include, for example, amorphous calcium compounds. Amorphous calcium compounds such as amorphous calcium phosphate (ACP), amorphous calcium phosphate fluoride (ACPF), amorphous calcium carbonate phosphate (ACCP), amorphous calcium carbonate phosphate (ACCP), and amorphous calcium carbonate phosphate fluoride (ACCPF) may be used as a sensitivity relief agent alone or in combination with alkali metal salts. These amorphous compounds are disclosed in U.S. Pat. Nos. 5,037,639, 5,268,167, 5,437,857, 5,562,895, 6,000,341, and 6,056,930, the disclosure of each is hereby incorporated by reference in its entirety.

Other alkali metal salts may include amorphous strontium compounds such as amorphous strontium phosphate (ASP), amorphous strontium phosphate fluoride (ASPF), amorphous strontium calcium phosphate (ASCP), amorphous strontium calcium carbonate phosphate (ASCCP), amorphous strontium carbonate phosphate fluoride (ASCPF) and amorphous strontium calcium carbonate phosphate fluoride (ASCCPF). These compounds are disclosed in U.S. Pat. No. 5,534,244, the content of which is hereby incorporated by reference in its entirety.

The components of the amorphous compounds are generally present separately, for example, a first component may include a source of phosphate and a second component may include a source of calcium or strontium. When the two components are mixed, the source of phosphate and the source of calcium, strontium or mixture may combine to form amorphous calcium phosphate, which when applied to teeth, may precipitate onto the surface of the teeth where it may be incorporated into hydroxyapatite, assisting in remineralization of the tooth enamel, as well as decreasing sensitivity. Thus, these alkaline metal salts also have the added advantage of being used in remineralization.

The source of phosphate may be, for example, present in an amount of from about 0.2% to about 5% by weight, more for example, between about 0.2% to about 4% by weight.

The source of calcium, strontium or combinations thereof may be present in an amount of, for example, from about 0.25% by weight to about 1.5% by weight, more for example, about 0.3% to about 1% by weight.

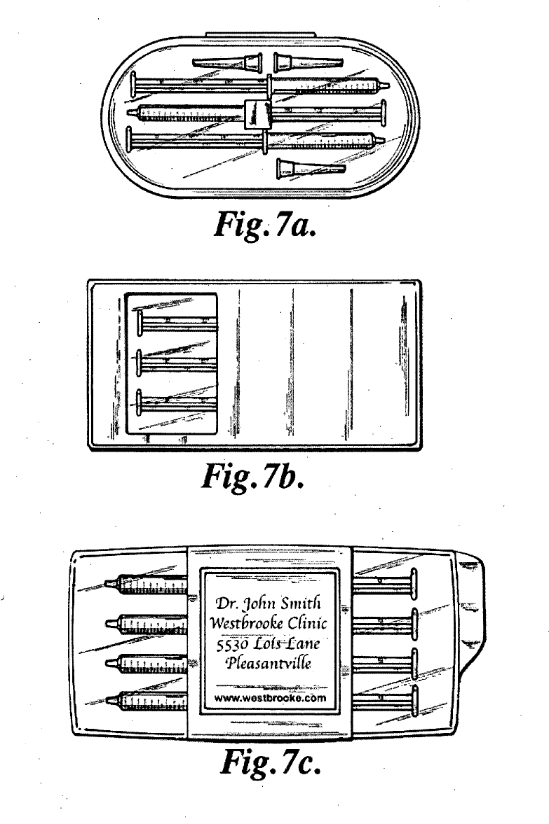

In one embodiment, the gels containing amorphous compounds may be present in separate compartments of a container, such as a dual-barrel syringe, such as shown in FIGS. 7a-c. Even though the source of calcium or strontium is kept separately from the source of phosphate, a separate container or compartment is not the only way to effect separation. In another embodiment, the components may be present in the same compartment, and separation may be effected by means of distance, or a partition which may involve encapsulating the source of calcium or strontium in one capsule, layer or an immobilized medium, generally referred to as a component, and the source of phosphate in another capsule, layer, an immobilized medium, or phase separated, also generally referred to as a component.

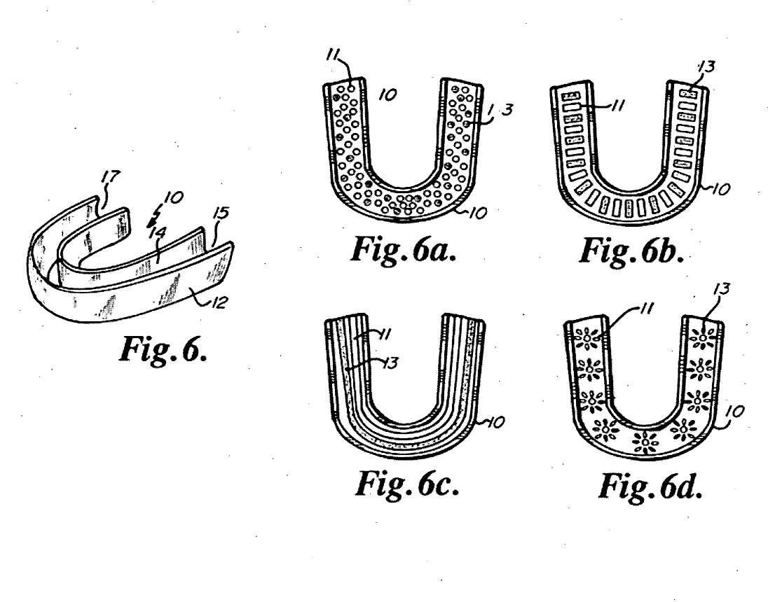

In yet another embodiment, the components may be contained in a tray, for example, coated separately in a tray, as shown in FIG. 6, and that the components are physically separated prior to use. Some of these embodiments are shown in FIGS. 6a-6d.

Multiple separated coatings may be used to keep the components separate prior to use. One set of coatings may contain a calcium source, such as calcium nitrate, and another set of coatings may contain a phosphate source, such as sodium phosphate. For example, a set of coating stripes, spots, checkerboards, floral designs, pictures or other shape, may contain parts of an amorphous calcium phosphate (ACP) or amorphous strontium phosphate composition.

In one embodiment, a water soluble carrier such as pullulan may be used to form the coating. Pullulan is a soluble polysaccharide polymer derived from a fungal organism composed of triose sugar units. Pullulan coatings may be a self adherent, flexible and/or relatively high strength film that may resiliently remain on the applied surface(s) and that dissolves very quickly on contact with a wet or moist environment. They are also useful in encapsulating other materials, as pullulan has oxygen barrier properties and is thus further useful for preserving the encapsulated material(s).

The coating may cover any desired portion of the device. In general, the coating may cover at least a portion that may be in contact with the teeth or oral tissues when in use. The amorphous compounds are formed when the coatings dissolve due to oral cavity moisture and release the components of the composition to combine.

In another embodiment, when the other components of the sensitivity relief gel is introduced to the tray already including separate sets of coatings of components of amorphous calcium or strontium phosphate, the amorphous compounds are formed.

Fluorides are effective in treating dental caries and may include metal fluoride salts such as sodium fluoride, stannous fluoride, sodium monofluorophosphate, potassium fluoride, lithium fluoride, ammonium fluoride, zinc ammonium fluoride, tin ammonium fluoride, calcium fluoride, cobalt ammonium fluoride, and water soluble amine hydrofluorides.

Some of these alkaline metal salts may also be used in fluoridating teeth. All of the above amorphous compounds which form the amorphous compounds, when applied either onto or into dental tissue, may help to prevent and/or repair dental weaknesses such as dental caries, exposed roots and dentin sensitivity, as discussed above.

It is well recognized in the art that the efficacy of the fluoride treatment depends on the solubility of the fluoride compound, and hence, the availability of fluoride ions, and the primary method of testing for fluoride efficacy in a clinical sense is to measure “total” and “available” fluoride. Thus, a fluoride source, for example, generally includes soluble fluoride including sodium fluoride, potassium fluoride, stannous fluoride or mixtures thereof.

At the same time, though acidic fluoride solutions are amongst the most effective ones in delivering fluoride ions, they may also usually lead to demineralization of the teeth. The pH of the gel composition is thus generally kept at about 5.5 or above, more for example, between about 7 to about 8.5. The addition of some metal ions such as Indium III, however, have been shown to prevent demineralization at low pH by decreasing enamel solubility, thus expanding the range of possible pH to be used.

The addition of some alkaline metal salts, such as amorphous calcium and/or amorphous strontium salts, discussed above, may counter-act this demineralizing effect and provide remineralization.

Further, other sources of calcium and/or strontium, and/or phosphate salt, wherein the salt provides sustained release of calcium and/or strontium and/or phosphorous ions in the presence of water or saliva for forming hydroxyapatite and providing remineralization may be used. The salt may include a calcium or strontium salt of glycerophosphate, lactate, gluconate, fumarate, acetate, chloride, or mixtures thereof; tricalcium phosphate; dicalcium phosphate anhydrous; dicalcium phosphate dehydrate; octacalcium phosphate; tetracalcium phosphate; monocalcium phosphate; or mixtures thereof. The salt may be, for example, present in a particulate form having a particle size, for example, of less than about 50 um, and more for example, of from about 1 to about 20 um.

Further, a source of calcium and/or strontium may be used to provide sustained release of calcium and/or strontium ions in the presence of water or saliva, for forming hydroxyapatite and providing remineralization upon combination with a source of phosphate already present in the saliva or on the tooth or teeth.

Another example of an alkaline compound includes a sparingly soluble calcium phosphate salt in dry particle form, for example, α-tricalcium phosphate, β-tricalcium phosphate, tetracalcium phosphate, monocalcium phosphate monobasic, dicalcium phosphate anhydrous, dicalcium phosphate dihydrate, octacalcium phosphate or mixtures thereof.

More for example, in addition to the source of calcium and/or strontium salt, a separate source of phosphate salt in particulate form may be included, as noted above. These may be sparingly soluble or soluble and may include monosodium phosphate (NaH₂PO₄), disodium phosphate, tetrapotassium pyrophosphate or mixtures thereof.

In general, the exemplified ranges of pH may be achieved by including a pH adjusting agent in the composition. Exemplary adjusters include potassium hydroxide and hydrochloric acid. Others may include phosphates like tetrapotassium pyrophosophates, and nitrates.

The total fluoride of the compositions of the present invention may be, for example, between about 500 ppm to about 1500 ppm, more for example, and even more for example, from about 800 to about 1100 ppm.

The gelling matrix may be formed primarily with non-ionic surfactants, though other surfactants may also be present. Examples of non-ionic surfactants may include ethylene oxide copolymers, for example, those including both a hydrophilic component and a hydrophobic component, including copolymers of ethylene oxide and propylene oxide. The copolymers may be block copolymers of propylene oxide (hydrophobic component), and ethylene oxide (hydrophilic component). The propylene oxide block is generally sandwiched between two ethylene oxide blocks. Literally hundreds of versions of such non-ionic surfactants having both hydrophobic and hydrophilic blocks are possible by incremental alteration of both hydrophobe and hydrophile. In addition, heteric or alternating ethylene oxide/propylene oxide structures may be introduced internally or at the end of the molecule. Examples include Pluronic F-127, P-84, F-108, F-98, F-88, F-87 and mixtures thereof, available from BASF Corporation (North Mount Olive, N.J., USA).

Other gelling agents suitable for use may include, for example, other non-ionic surfactants, such as the classes that limit the number of available hydrophobes and effect changes in surfactant function only by altering the hydrophiles, are suitable.

The amount of surfactants present in the gel may determine whether a gel can or cannot be formed as well as the gel's stability. With too little gelling agent, no gel can be formed. With too much gelling agent, no gel formation is possible. Stable gels are formed when the surfactants are present within the appropriate range.

Exemplary percent of hydrophobic components in the gelling agents may range from about 10 to about 80, more for example, from about 30 to about 60, even more for example, from about 31 to about 38, and even more for example, from about 33 to about 36.

Also, the non-ionic surfactant may be present in an amount that ranges from about 15% to about 50% by weight, more for example, from about 20% to about 45% by weight of the gel, even more for example, from about 25% to about 35% of the gel.

The total molecular weight of the non-ionic surfactant may vary. Exemplary molecular weights may range from about 3000 to about 20,000, more for example, from about 5,000 to about 12,000.

In general, for example, for Pluronic® surfactants, the gelling tendencies of block copolymers increase with hydrophile content and with total molecular weight.

For example, the hydrophile content may vary from about at least 50% to about 80%, more for example, from about 60% to about 80%.

The molecular weights of the hydrophobe portion of the, for example, Pluronic® surfactants, for example, 127 or 108. may vary from at least about 2000 to about 4000, more for example, at least about 2500 to about 4000.

Surprisingly, the alkali salts present in the gel for their de-sensitizing effects, such as potassium nitrate or sodium saccharin, also act to stabilize the gel. Therefore, stability of the gels of the present invention may not be dependent on the addition of gel stabilizers.

For example, at least one insoluble stabilizer including vitamin E oil, anise, eugenol, natural mint flavors or mixtures thereof may be added to further help the stability of the gel formation, if desired.

These insoluble stabilizers may, for example, be present from about 1% to about 5%, more for example, from about 2% to about 4% by weight, in addition to the alkali metal salts.

Aliphatic polyols, such as glycerine, may also be added in an amount of about 0% to about 10%, more for example, from about 1% to about 4% by weight.

Water may also be added in an amount of from about 50% to about 70%, more for example, from about 55% to about 65%.

The gel is substantially free of any abrasives which is generally present in most dentrifices and may tend to cause irritation in a de-sensitizing gel.

There is a significant improvement in the handling characteristics of the gel from those of the prior art in that it is more easily applied by the dentist, and significantly more easily removed. Improved ease of use translates into faster procedures, reduced “office time” and a potential for reduced cost and increased profitability for the dental office. Without wishing to be bound by a theory, it is surmised that this advantage could be due to the increased wetting ability of the compositions of the present invention and reduced gumminess that may also indicate an increased availability of active ingredients and a possible improvement in overall efficacy.

The desirable gel properties may be correlated with modulus and/or viscosity measurements. In general, E′ (storage modulus) and E″ (loss modulus) of a composition, as well as the overall viscosity may be measured using a rheometer. E′, the storage modulus, can be thought of as the portion of the viscosity that comes from the elasticity of the sample. On the other hand, E″, the loss modulus, is the portion of viscosity that is primarily due to internal friction. E′ and E″ are also dependant on how fast the rotating disc (if such is used), oscillates (measured in Hertz). An oscillating frequency of about 0.01-1 Hz may roughly correlate with the equivalent of a gravitational force on a sample, for example, like paint or caulk on a wall. 10 Hz may roughly correlate with the action of chewing. A frequency of 50 Hz may roughly correlate with, for example, the impact of an object hitting the floor after being dropped, or more appropriately, the shear of being extruded through the tip of a syringe.

For a gel, for example, losing a large portion of its elastic modulus when going from lower frequencies to higher frequencies may translate into lower tendency to flow while at rest and higher tendency to flow more readily under impact. In practical terms, the above would mean a gel that flows more easily from the nozzle of a syringe, if such a device is used, or when being dispensed or squeezed out a tube, if such a device is used. At the same time, when applied to tooth or teeth, may tend to stay put or retain its shape and position. On the other hand, for a gel, for example, increasing its elasticity at higher frequencies may translate into lower tendency to flow under impact.

In one embodiment, gels of the present invention, the storage modulus (E′) may be, for example, at least about 250,000 Pa (Pascals) at low frequencies of about 0.01 Hz, at a strain of about 0% to about 10%, more for example, E′ may be at least about 350,000 Pa, and even more for example, E′ may be at least about 500,000.

The temperature of measurement may be between about 20° C. to about 40° C., more for example, from about 21° C. to about 37° C.

The gels of the present invention may have a storage modulus (E′) that, for example, stays substantially constant from a frequency of about 0.01 Hz to a frequency of about 50 Hz, at a strain of about 0% to about 10%. More for example, the storage modulus (E′) decreases from a higher value at about 0.01 Hz to a lower value at about 50 Hz. Even more for example, the storage modulus (E′) decreases from a higher value at about 0.01 Hz to a lower value of about 10% less at about 50 Hz; and still more for example, of about 15% less.

At the same time, a gel having higher storage viscosity value at lower frequencies may also translate into higher tendency to maintain its shape or stay put once applied.

The gels of the present invention may also have a storage viscosity of, for example, more than about 20,000 Pa·s (Pascal·seconds) at frequencies of about 0.1-0.5 Hz and at a strain of about 0% to about 10%, more for example, more than about 50,000 Pa·s, even more for example, 100,000 Pa·s.

The de-sensitizing gel may be prepared using any known method. Typically, in preparing the gel, the stabilizing polyols such as glycerin, and water sufficient to produce the desired concentration of the gel are completely mixed, before any salts, such as sodium fluoride, or potassium nitrate, are added and mixed at high speed until completely dissolved. A gelling agent such as PLURONIC® F-127 is added also at high speed until the gelling agent is completely dispersed and the resulting composition is almost like a whipped cream in texture.

Eugenol, sodium saccharin, and/or natural mint flavorings, for example, in undiluted form, are next added, resulting in a thickening of the bleaching gel. The gel may be mixed at ambient temperatures for about thirty to forty minutes, or until the desired consistency is achieved, then vacuum degassed.

Alternatively, following addition of eugenol and/or mint flavorings, the gel can be stored at temperatures sufficiently low to liquify the gelling agent, for example, at approximately 10° F. when PLURONIC® F-127 is used, allowing air trapped in the gel to rise to the surface, where it may be scrapped off as foam or removed by vacuum degassing.

The whitening composition may include any commercial whitening composition including Zoom®!gel, Zoom®! Turbo, Zoom® 2 gel, BriteSmile® Procedure Gel; BriteSmile® Take Home Gel; and others, all available from Discus Dental, Inc. or BriteSmile Professional, Inc., Culver City, Calif. Some of these gels are capable of whitening without the assistance of heat and/or light. Examples include NiteWhite®, DayWhite®, NiteWhite® Turbo, NiteWhite® ACP, Nitewhite® ACP Turbo, DayWhite® ACP, WhiteSpeed® JumpStart, Zoom Weekender®, BriteSmile®-to-go or BTG Pen, also available from Discus Dental, Inc. or BriteSmile Professional, Inc., Culver City, Calif. Some of these are described in more detail in U.S. Pat. Nos. 5,908,614, 6,576,227; 6,221,341; 6,488,914 5,922,307; 6,331,292; 6,986,883; U.S. publication nos. 2003/211055; 2004/0101497; 2005/0008584; 2005/0026107; 2004/0146467; U.S. application Ser. No. 11/271,283 entitled, “Dental Whitening Systems” filed on Nov. 9, 2005; Ser. No. 11/271,412 entitled, “Dental Whitening Compositions” filed Nov. 9, 2005; Ser. No. 11/288,504 entitled, “Dental Whitening Compositions,” filed Nov. 28, 2005, the contents of all of these are incorporated herein by reference.

Some of these whitening gels may be light and/or heat activated gels and include those described in U.S. Pat. Nos. 5,645,428; 5,713,738; 6,258,388; 6,361,320; 6,162,055; 6,343,933; 6,416,319; 6,958,144; U.S. publication nos. 2002/0137001; 2002/0141951; 2003/0198605; 2003/0089886; 2005/0084825; 2005/0265933; 2005/0048434; U.S. application Ser. No. 11/271,283 entitled, “Dental Whitening Systems” filed on Nov. 9, 2005; the contents of all of these are incorporated herein by reference.

In one aspect, the whitening composition may be packaged in a separate container or package inside a main package. The container or package may include a syringe, a double-barrel syringe, a squeezable tube, a pumpable container, a multiple-compartment container, or similar. Some examples of containers are found in U.S. Pat. Nos. 6,312,670; 6,488,914; 6,322,773; 6,322,773; 6,514,543; 6,536,628; 5,819,988; 6,065,645; 6,394,314; 3,564,972, and 6,698,622, the contents of all of these are incorporated herein by reference.

In another aspect, the package may contain more than one whitening package. For example, the package may include multiple containers to provide for multiple uses.

In a further aspect, the viscous gel may be packaged in a separate container including a syringe, a double-barrel syringe, a squeezable tube, a pumpable container, a multiple-compartment container, or similar.

In one embodiment, the package may be a bubble-like container, an example of which is shown in FIG. 7a. This type of package may be cast, molded or injection molded using any moldable polymeric material including polyethylene, polypropylene, polyester, polystyrene, ethylene-vinylacetate, or similar. In another embodiment, the package may be a box-like container, an example of which is shown in FIG. 7b. In one aspect, the container may be fitted with a band having personalization capability, as shown in FIG. 7c. The personal information may be imprinted on the band, printed on the band, or heat molded on the band. In another aspect, the container may be fitted with a band capable of different color schemes.

A non-limiting example of the de-sensitizing gel is provided in accordance with practice of the present invention as follows:

### Gel 1

| Material | Trade Name | % | Grams |
|---|---|---|---|
| Water | | 62.66 | 125.32 |
| Glycerin | | 2.00 | 4 |
| KNO3 | | 5.00 | 10 |
| Sodium Fluoride | | 0.243 | 0.486 |
| Sodium Saccharin | | 0.25 | 0.5 |
| Poloxomer 407 | Pluronic F-127 | 28.00 | 56 |
| Eugenol | | 0.75 | 1.5 |
| Peppermint Flavor | | 1.10 | 2.2 |
| Total | | 100.00 | |

A non-limiting examples of the de-sensitizing gel in two components with ACP is provided in accordance with practice of the present invention as follows:

### Gel 2

Calcium ACP Gel:

| Material | Trade Name | % |
|---|---|---|
| Water | | 71.85 |
| Potassium Nitrate | | 5 |
| Calcium Nitrate | | 0.5 |
| Sodium Saccharin | | 0.25 |
| Poloxamer 338 | Pluronic F-108 | 20 |
| Natural Peppermint | Oil PE-05523 | 2.4 |
| Total | | 100.00 |

Phosphate ACP Gel:

| Material | Trade Name | % |
|---|---|---|
| Water | | 72.88 |
| Potassium Nitrate | | 5 |
| Na₂HPO₄ | | 0.25 |
| NaH₂PO₄ | | 0.25 |
| Sodium Fluoride | | 0.442 |
| Sodium Saccharin | | 0.25 |
| Poloxamer 338 | Pluronic F-108 | 20 |
| Natural Peppermint | Oil PE-05523 | 0.93 |
| Total | | 100.00 |

As discussed above, the source of calcium and the source of phosphate are kept apart in separate compartments for amorphous calcium phosphate.

A gel may be tested according to the following method:

Test Method

The following test was carried out using an oscillating rheometer, as exemplified in FIGS. 5a and 5b.

The device used consisted of two circular discs, 10a and 10b, stacked on top of each other with a small gap between them: 10a being fixed and 10b being movable. A sample of Gel 1 100 was placed in the gap 10c between the disc 10a and the movable disc 10b. The movable disc 10b was then set to oscillate back and forth at a set frequency scan or sweep.

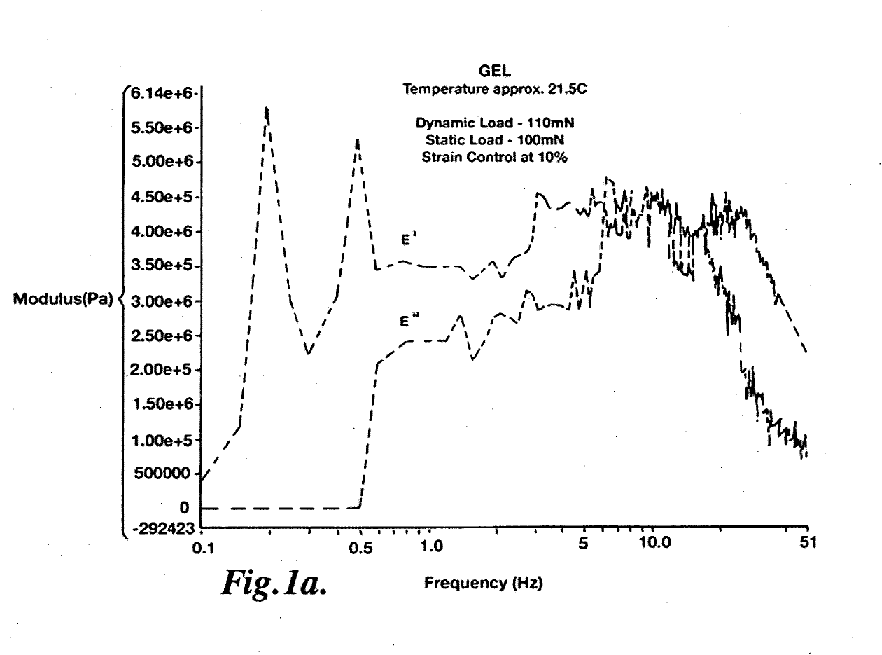

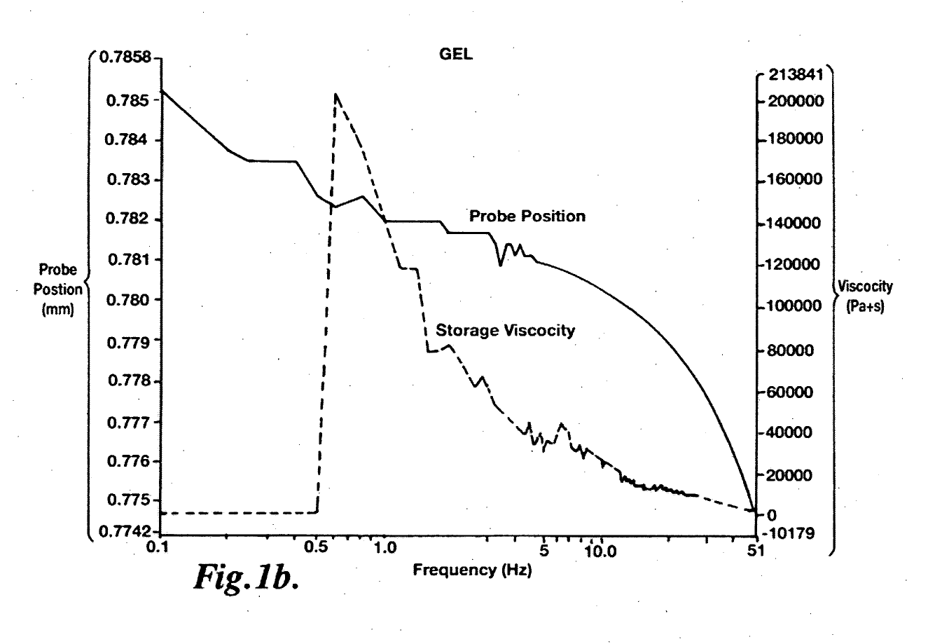

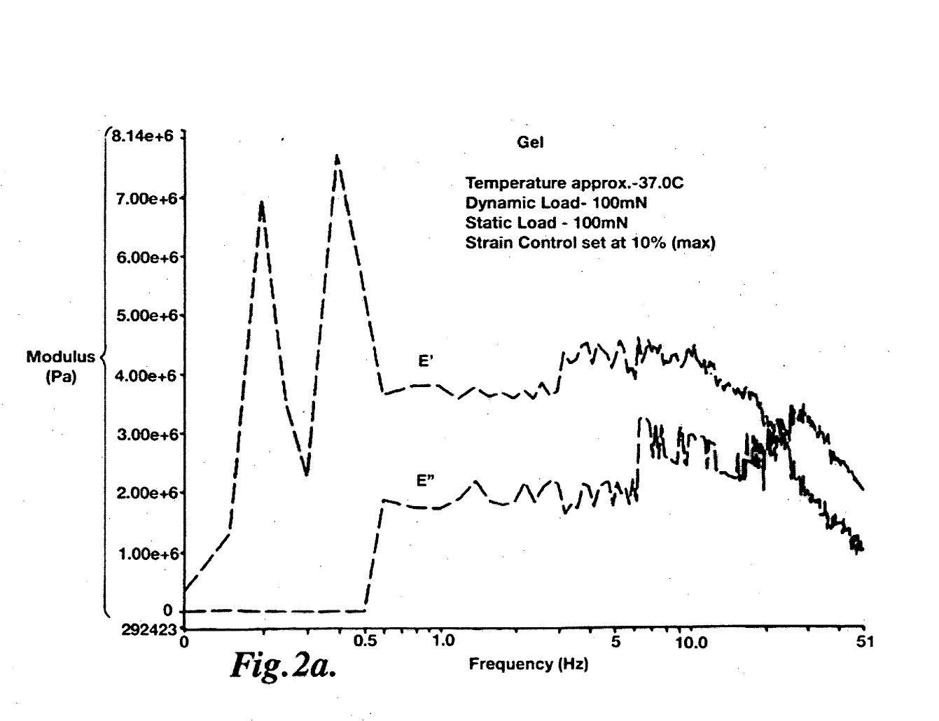

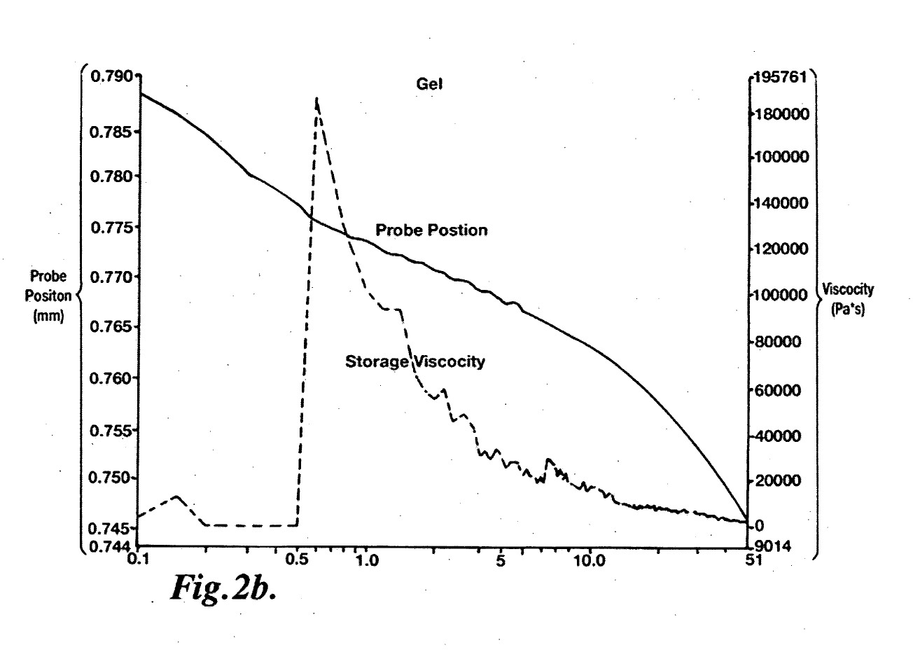

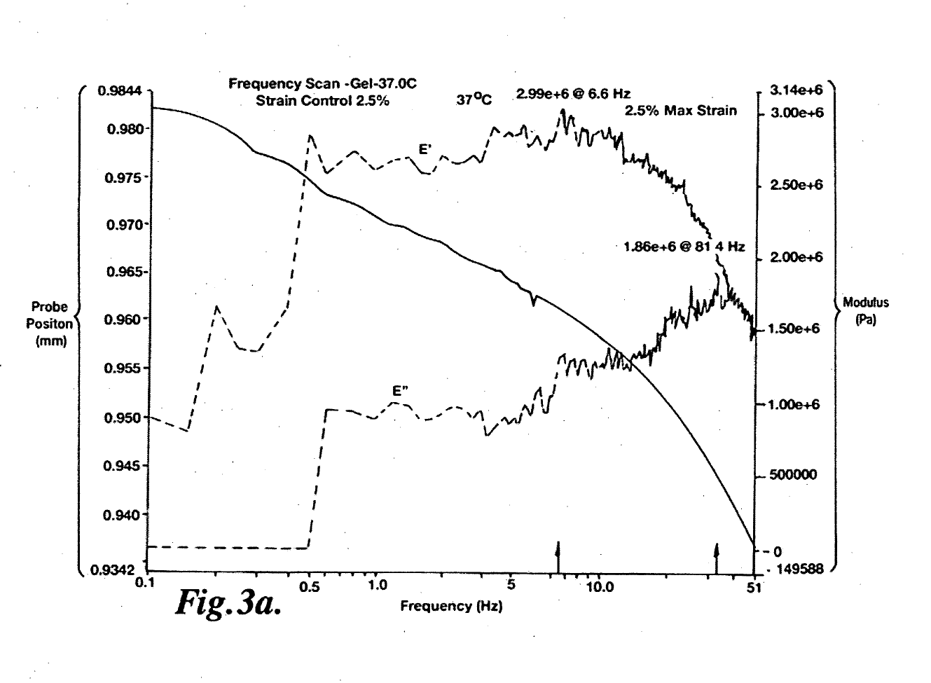

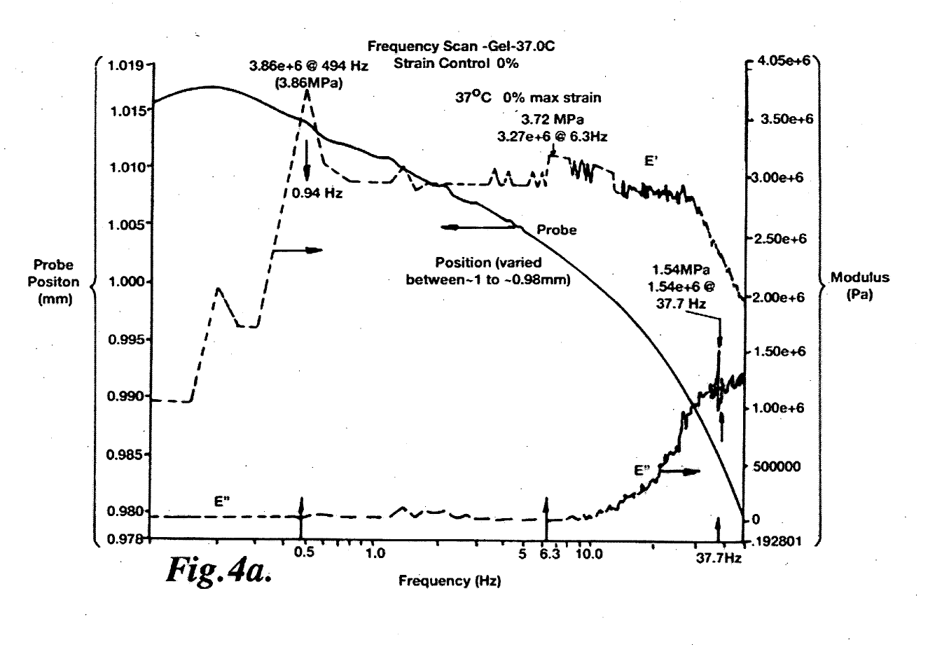

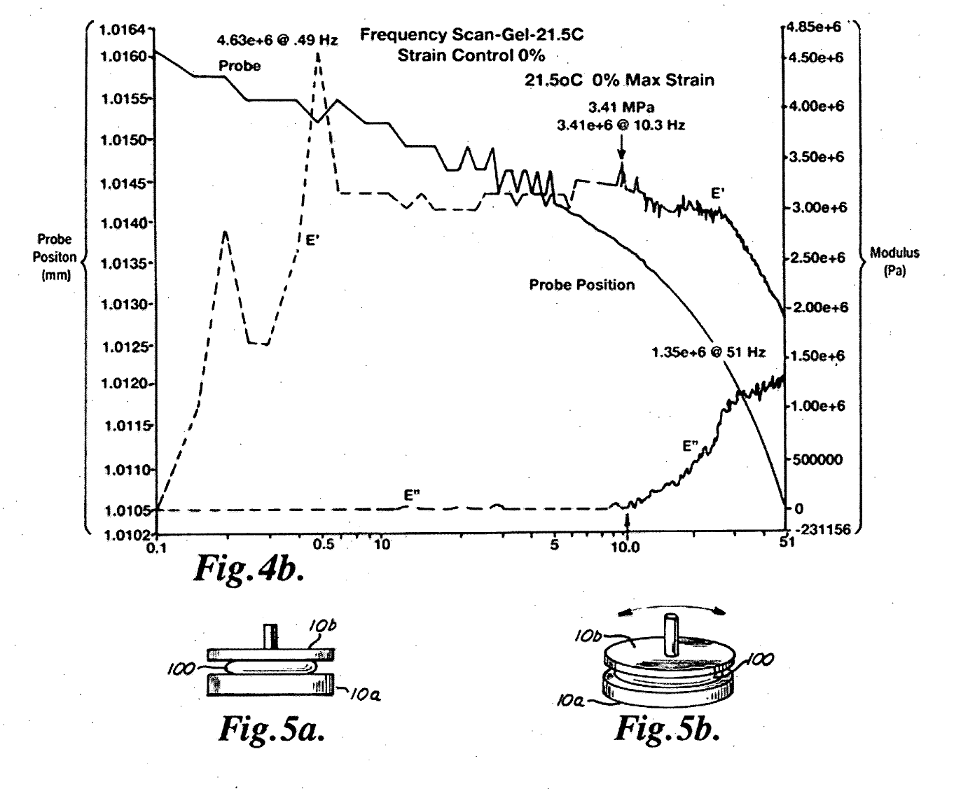

A strain % was also set and for the measurements made, at 0%, 2.5% and 10%, respectively, as shown in FIGS. 1-4.

The temperature of measurements was set at 21.5° C. and 37° C., as shown in FIGS. 1-4.

The gel may be applied generally, for example, after a whitening session, either at the dentist office or at home. It may be applied directly to a patient's teeth for a clinically relevant time period, usually 5-15 minutes and then suctioned off. An alternative means of application is by means of “night guard” form fitting tooth trays.

The method of application thus includes:

providing a bleaching gel including at least one peroxide to effect teeth bleaching; and applying a sensitivity relief gel of the present invention.

For example, the gel may be applied after a teeth whitening session at the dentist office. Exemplary methods of using a whitening equipment and process include those involving the use of light to activate the whitening compositions. One exemplary method is described in detail in U.S. Pat. No. 7,086,862, entitled “Tooth Bleaching Process”, the entire contents of which are hereby incorporated by reference.

Other whitening equipment include those disclosed in U.S. Issued Pat. No. 6,162,055, 6,343,933, 6,416,319, 6,213,671; and U.S. patent applications U.S. Ser. No. 11/173,839, entitled “Illumination System”; U.S. Ser. No. 11/179,736, entitled “Light Activated Tooth Whitening”; U.S. Ser. No. 10/958,058 “Apparatus For Simultaneous Illumination of Teeth”; U.S. Ser. No. 11/197,868 entitled, “Apparatus For Simultaneous Illumination of Teeth”; U.S. Ser. No. 10/434,597 entitled, “Light-Activated Tooth Whitening Composition And Method Of Using Same”; the entire contents of which are hereby incorporated by reference.

Briefly, the first stage of the method in accordance with aspects of the present invention involves isolation of the teeth and protection of the non-tooth surfaces that might otherwise be exposed to the whitening composition in the absence of protection. Optionally, a commercially available protective lip cream may be applied to the lips to protect the lips. A cotton swab may be used to coat the lips with the cream. The cream is configured to keep the lips moist during the procedure and provide added protection from light exposure. Exemplary protective lip creams include paba free creams with high SPF rating, of about 30 or higher.

Next, a lip retractor, such as the Zoom®!™ Retractor, or a SeeMore™ Retractor, both available from Discus Dental, Inc., of Culver City, Calif., is installed to pull the lips away from the teeth; or ones described in U.S. Pat. Nos. D504,721 and 6,923,761, and U.S. patent application Ser. No, 11/173,297, entitled, “Retracting Devices”; U.S. Ser. No. 11/203,540, entitled, “Retracting Devices”, the contents of which are hereby incorporated by reference. For additional isolation, a dentist can optionally apply medical grade petroleum jelly in the upper and lower vestibules, and cover the area with 2″×2″ four-ply gauze squares (Banta Healthcare Group), cotton rolls, or other suitable materials having suitable size to cover any remaining exposed tissues within the oral cavity.

A protective material may also be applied to the gingiva to protect the gums from exposure to the whitening composition and the light radiation to be applied thereon. For example, a light-cured dental resin, such as Discus Dental's Liquidam™ Dental Dam, Culver City, Calif., may be applied and cured. The gingiva is then, for example, dried prior to application of the protective material. The protective material, which is generally a light curable resin-based material, may be syringed directly onto the gingiva with sufficient amount for full gingival protection. The application may extend distally for at least one tooth beyond the area to receive the whitening application. The application may also extend up or down to meet the gauze or retractor cover to protect the margins. Once the application of the dental dam is complete, the margins are rechecked to ensure that the dam is sealed against the enamel to prevent leakage and oxidation of tissue during the whitening procedure.

The whitening gel may be applied directly to the surface of the teeth as, for example, from the tip of a dual barrel syringe, such as that disclosed in U.S. Pat. Nos. 5,819,988, 6,065,645, 6,394,314, 6,564,972 and 6,698,622, the contents of which are incorporated herein by reference.

The gel may be applied to an approximate depth of about 1-2 mm. A brush may be used to ensure proper placement of the gel. Alternatively, the bleaching composition may be dispensed into a dappen dish or into a mixing pad for placement onto the tooth surface with a brush. If contact occurs between the whitening gel and tissue during application of the whitening composition, Vitamin E Oil may be applied to the oxidized tissue. The oil material will help sooth the tissue and create a barrier so that the whitening procedure can continue.

Once the whitening gel is applied to the teeth, a lamp, such as a short arc metal halide lamp, is directed at the patient's smile zone to illuminate the whitening gel and thereby activate the whitening process. An exemplary suitable lamp is the SMR-150UV1, Model No. 04-1001, from

Ushio America, Inc., of Cypress, Calif.; the ones disclosed in U.S. Pat. Nos. 6,162,055, 6,343,933, 6,416,319, 6,213,671 and 7,086,862; and U.S. patent applications U.S. Ser. No. 11/173,839, entitled “Illumination System”; U.S. Ser. No. 11/179,736, entitled “Light Activated Tooth Whitening”; U.S. Ser. No. 10/958,058 “Apparatus For Simultaneous Illumination of Teeth”; U.S. Ser. No. 11/197,868 entitled, “Apparatus For Simultaneous Illumination of Teeth”; U.S. Ser. No. 10/434,597 entitled, “Light-Activated Tooth Whitening Composition And Method Of Using Same”, the contents of all of which are hereby incorporated by reference.

For example, filters may be placed in front of the lamp to screen out unwanted or harmful wavelengths. Suitable filters include a UV filter (available from Optical Industrial of Houston, Tex., Part No. 03-1013), an IR filter (available from Swift Glass of Elmira, N.Y., part No. 03-1017), and/or a diffuser filter (available from Edmund Industrial Optics of Barrington, N.J., part No. 03-1020).

The IR filter may also be selected to filter all IR wavelengths to thereby reduce heat emitted to the patient. The UV filter is selected to remove some, but not all, of the ultraviolet wavelengths. For example, UV wavelengths below about 345 nm are filtered by the UV filter and only about 10-15% of wavelengths above about 400 nm are allowed to pass through the filter. The diffuser filter may be configured to diffuse the light rays and minimize the intensity of light irradiating onto the target area of the mouth. In one exemplary embodiment, the edge of the lamp may be positioned about 118 mm from the diffuser filter.

Once the lamp is secured in place and the smile zone illuminated, the whitening cycle can begin by activating the light. For the compositions of the instant invention, this may involve an approximately 10- to 20-minute, preferably 15-minute, cycle. If the light assembly has not been used recently, a warm up cycle of a few minutes may first be required. At the end of the cycle, the gel should be suctioned from the patient's teeth and any remaining gel removed with a dampen gauze.

Sometimes, a second and a third application of the whitening gel and light activating cycle are preferred. However, more cycles may be used if necessary to achieve the desired degree of whitening.

After the final cycle has been completed, the lamp is removed and the remaining whitening gel is suctioned and wiped from the teeth. The isolation materials (gauze, cotton rolls, gingival protection) are then removed and, with the retractor in place, the oral cavity is thoroughly rinsed and suctioned. Dental floss can be used to remove any dental resin material that remains interproximally.

With the retractor still in place, various post-treatment procedures may be optionally be carried out. For example, the de-sensitizing gel of the present invention may be applied, for example, by syringing it directly onto the teeth, approximately 2-3 mm thick, and allowing it to remain on the teeth for about 5 minutes before suctioning and rinsing.

In some embodiments, a dental device may be utilized to introduce a composition to the teeth. The dental device may be a tray 10, as shown in FIG. 6. The tray 10 may generally include a wall 12, a channel 14 to accommodate a user's arch, and open ends 15, 17 to accommodate different arch dimensions.

In an exemplary embodiment, multiple separated coatings may be used to contain therapeutic agents that may be kept separate prior to use. For example, a set of coating stripes, spots or other shape 11, 13 may contain parts of a remineralizing composition, such as an amorphous calcium phosphate (ACP) or amorphous strontium phosphate composition, as shown in FIGS. 6a, 6b, 6c and 6d. One set of coatings 11 may contain a calcium or strontium source, such as calcium nitrate, and another set of coatings 13 may contain a phosphate source, such as sodium phosphate. The composition may then be formed when the coatings dissolve due to oral cavity moisture and release the components of the composition to combine.

In one embodiment, a one component water containing composition may be introduced to the tray already including separate sets of coatings of components of amorphous calcium or strontium phosphate.

The coatings 11, 13 may include a water soluble carrier in which at least one chemical agent may be dispersed. The water soluble carrier may be, for example, a soluble polymeric material. Such materials include, but are not limited to, hydrophilic materials including polysaccharides such as carrageenan, chondroitin sulfate, glucosamine, pullulan, soluble cellulose derivatives such as hydroxypropyl cellulose and hydroxymethyl cellulose, polyacrylic acid, polyvinyl alcohol, polyethylene glycol (PEG), polyethylene oxide (PEO), ethylene oxide-propylene oxide co-polymer, polyvinylpyrrolidone (PVP) and/or any other appropriate material. In an exemplary embodiment, the water soluble carrier included is pullulan.

The coatings 11, 13 may cover any desired portion of the tray 10. In general, the coatings 11, 13 may cover at least a portion that may be in contact with the teeth or oral tissues when in use.

In some embodiments, coatings 11, 13 may be disposed in patterns. The coatings 11, 13 may be formed to give visual appeal to the user, such as by introducing particular patterns. Patterns may include, for example, stripes, as shown in FIGS. 6b, 6c, spots, as shown in FIG. 6a, floral designs, as shown in FIG. 6d, pictures, and/or any other appropriate pattern.

While this invention is described in detail with reference to a certain exemplified embodiments, it should be appreciated that the present invention is not limited to those precise embodiments. Rather, in view of the present disclosure which describes the current best mode for practicing the invention, many modifications and variations would present themselves to those of skill in the art without departing from the scope and spirit of this invention. In particular, it is to be understood that this invention is not limited to the particular methodology, protocols, and reagents described as such may vary, as will be appreciated by one of skill in the art.

1. A sensitivity relief gel comprises:
from about 2.5% to about 6% by weight of at least one de-sensitizing substance;
at least one source of fluoride ion at a concentration of up to about 1500 ppm; and
at least one non-ionic surfactant;
wherein said gel has a storage modulus (E′) of about at least 250,000 Pa (Pascals) at low frequencies of about 0.01 Hz, at a strain of about 0% to about 10%.

2. The sensitivity relief gel of claim 1 wherein said gel a storage modulus (E′) of about at least 350,000 Pa (Pascals) at low frequencies of about 0.01 Hz, at a strain of about 0% to about 10%.

3. The sensitivity relief gel of claim 1 wherein said gel has a storage modulus (E′) of about at least 500,000 Pa (Pascals) at low frequencies of about 0.01 Hz, at a strain of about 0% to about 10%.

4. The sensitivity relief gel of claim 1 wherein said gel is substantially free of abrasives.

5. The sensitivity relief gel of claim 1 wherein said non-ionic surfactant comprises a block copolymer with hydrophobic and hydrophilic blocks.

6. The sensitivity relief gel of claim 1 wherein said non-ionic surfactant comprises an ethylene oxide block and a propylene oxide block.

7. The sensitivity relief gel of claim 1 wherein said non-ionic surfactant is present from about 20% to about 45% by weight in the gel.

8. The sensitivity relief gel of claim 1 wherein said de-sensitizing substance comprises an alkali metal salt, an alkaline metal salt, or mixtures thereof.

9. The sensitivity relief gel of claim 1 wherein said de-sensitizing substance is selected from the group consisting of soluble lithium salts, sodium salts, potassium salts, amorphous calcium salts, amorphous strontium salts, sparingly soluble calcium phosphate solids and mixtures thereof.

10. The sensitivity relief gel of claim 8 wherein said alkaline metal salt is selected from the group consisting of amorphous calcium phosphate (ACP), amorphous calcium phosphate fluoride (ACPF), amorphous calcium carbonate phosphate (ACCP), amorphous calcium carbonate phosphate (ACCP), amorphous calcium carbonate phosphate fluoride (ACCPF), amorphous strontium phosphate (ASP), amorphous strontium phosphate fluoride (ASPF), amorphous strontium calcium phosphate (ASCP), amorphous strontium calcium carbonate phosphate (ASCCP), amorphous strontium carbonate phosphate fluoride (ASCPF), amorphous strontium calcium carbonate phosphate fluoride (ASCCPF), α-tricalcium phosphate, β-tricalcium phosphate, tetracalcium phosphate, monocalcium phosphate monobasic, dicalcium phosphate anhydrous, dicalcium phosphate dihydrate, octacalcium phosphate and mixtures thereof.

11. The sensitivity relief gel of claim 1 wherein said de-sensitizing substance comprises a mixture of potassium nitrate and amorphous calcium phosphate.

12. The sensitivity relief gel of claim 1 wherein said source of fluoride is selected from the group consisting of sodium fluoride, stannous fluoride, sodium monofluorophosphate, potassium fluoride, lithium fluoride, ammonium fluoride, zinc ammonium fluoride, tin ammonium fluoride, calcium fluoride, cobalt ammonium fluoride, water soluble amine hydrofluorides and mixtures thereof.

13. The sensitivity relief gel of claim 1 wherein said fluoride ion is present from about 800 to about 1100 ppm.

14. A sensitivity relief gel comprising:
at least one de-sensitizing substance comprising a mixture of an alkali metal salt and an amorphous compound;
at least one source of fluoride ion at a concentration of up to about 1500 ppm; and
at least one non-ionic surfactant;
wherein said gel has a storage viscosity of more than about 20,000 Pa·s (Pascal·seconds) at frequencies of about 0.1-0.5 Hz, at a strain of about 0% to about 10%.

15. The sensitivity relief gel of claim 14 wherein said gel has a storage modulus (E′) of about at least 250,000 Pa (Pascals) at low frequencies of about 0.01 Hz, at a strain of about 0% to about 10%.

16. The sensitivity relief gel of claim 14 wherein said non-ionic surfactant is present from about 15% to about 50% by weight in the gel.

17. The sensitivity relief gel of claim 14 wherein said gel has a storage modulus (E′) that is substantially constant from a frequency of about 0.01 Hz to a frequency of about 50 Hz, at a strain of about 0% to about 10%.

18. The sensitivity relief gel of claim 14 wherein said non-ionic surfactant comprises a block copolymer with hydrophobic blocks of propylene oxide and hydrophilic blocks of ethylene oxide.

19. The sensitivity relief gel of claim 14 wherein said non-ionic surfactant is present from about 25% to about 35% by weight in the gel.

20. The sensitivity relief gel of claim 14 wherein said alkali metal salt is present from about 3 to about 5% by weight in the gel.

21. The sensitivity relief gel of claim 14 wherein said gel is packaged in a two-compartment vessel.

22. The sensitivity relief gel of claim 14 wherein said alkali metal salt is selected from the group consisting of potassium nitrate, sodium saccharin and mixtures thereof.

23. The sensitivity relief gel of claim 14 wherein said alkaline metal salt is selected from the group consisting of amorphous calcium phosphate (ACP), amorphous calcium phosphate fluoride (ACPF), amorphous calcium carbonate phosphate (ACCP), amorphous calcium carbonate phosphate (ACCP), amorphous calcium carbonate phosphate fluoride (ACCPF), amorphous strontium phosphate (ASP), amorphous strontium phosphate fluoride (ASPF), amorphous strontium calcium phosphate (ASCP), amorphous strontium calcium carbonate phosphate (ASCCP), amorphous strontium carbonate phosphate fluoride (ASCPF), amorphous strontium calcium carbonate phosphate fluoride (ASCCPF), and mixtures thereof.

24. The sensitivity relief gel of claim 14 wherein said amorphous compound comprises at least two-components separated from each other prior to use.

25. A sensitivity relief gel comprising:
at least one sensitivity relief substance;
at least one source of fluoride ion;
at least one non-ionic surfactant;
wherein said gel has a storage modulus of about at least 250,000 Pa (Pascals) at low frequencies of about 0.01 Hz, at a strain of about 0% to about 10%, said storage modulus being substantially constant from a frequency of about 0.01 Hz to a frequency of about 50 Hz, at a strain of about 0% to about 10%.

26. The sensitivity relief gel of claim 25 wherein said fluoride ion is present at a concentration from about 800 to about 1500 ppm.

27. The sensitivity relief gel of claim 25 wherein said non-ionic surfactant is present from about 15% to about 50% by weight in the gel.

28. The sensitivity relief gel of claim 25 wherein said non-ionic surfactant comprises a block copolymer with hydrophobic blocks of propylene oxide and hydrophilic blocks of ethylene oxide.

29. The sensitivity relief gel of claim 25 wherein said de-sensitizing substance is selected from the group consisting of soluble lithium salts, sodium salts, potassium salts, amorphous calcium salts, amorphous strontium salts, sparingly soluble calcium phosphate solids, and mixtures thereof.

30. The sensitivity relief gel of claim 25 wherein said gel has a storage viscosity of more than about 20,000 Pass (Pascal·seconds) at frequencies of about 0.1-0.5 Hz, at a strain of about 0% to about 10%.
